(19) United States
(12) Patent Application Publication
(10) Pub. No.: US 2010/0143201 A1
(43) Pub. Date: Jun. 10, 2010

(54) MAINTENANCE FREE OXIDIZER GENERATION AND APPLICATIO DEVICE FOR ENVIRONMENTAL MICROBIOLOGICAL CONTROL

(76) Inventors: Ronald B. Long, Wickenburg, AZ (US); Robert E. Varga, Wickenburg, AZ (US); Dodie K. Long, Wickenburg, AZ (US)

Correspondence Address: BROWDY AND NEIMARK, P.L.L.C.;624 NINTH STREET, NW, SUITE 300, WASHINGTON, DC, 20001-5303, US

(21) Appl. No.: 12/532,286

(22) PCT Filed: Mar. 21, 2008
(86) PCT No.: PCT/US08/57931

§ 371 Date: Feb. 12, 2010

Related U.S. Application Data
(60) Provisional application No. 60/896,258, filed on Mar. 21, 2007.

(51) Int. Cl. B01J 19/12; A23L 3/358; A61L 2/18

(52) U.S. Cl. 422/111

(57) ABSTRACT

A food and surface sanitizing apparatus has a compact corona discharge type ozone generator, having a permanently sealed small diameter, concentric anode, dielectric and cathode set which does not require dry air feed and which requires no maintenance. A dedicated cooling apparatus permits consistent ozone output over extended time of use. The apparatus is encapsulated in a housing along with an apparatus for dissolving a portion of the ozone in the stream of water passing through an arrangement of tubing. A sensor recognizes the presence of oxidizer in the water stream and adjusts the output of the ozone generator to maintain a predetermined level of dissolved ozone. A water flow quantifier enumerates the total volume of water processed.

### FIELD

The present invention relates to ozone generators employing corona discharge technology. More particularly, the present invention relates to corona discharge ozone generators which require no maintenance. Further, the present invention relates to corona discharge ozone generators embodied within an aqueous ozone application apparatus which possesses a means of sensing and communicating oxidizer presence in solution.

### BACKGROUND

The history and use of ozone as a water purifier is well known. More recently, the application of ozone as a food and surface sanitizer has been documented, including laboratory work by the present inventor with such institutions as the Food Science and Nutrition Department of California Polytechnic University (San Luis Obispo, Calif.) and the Department of Animal Science, Clemson University. These studies confirmed that very small amounts of ozone, <0.5 Parts Per Million, in brief contact with the surfaces of foods, such as produce, meat & poultry, can effectively reduce the dangers associated with foodborne pathogens by more than four logs (99.99%).

In July, 2001, the United States Food and Drug Administration approved the use of ozone in both gas phase and aqueous phase for contact with all foods. This official approval opened the door for the advent of a variety of ozone based apparatuses for use by food processors. The FDA approval also opened the door to the application of ozone for food and surface sanitizing for point of use by food service establishments and consumers.

However, an effective, low maintenance, small, corona discharge ozone generator that reliably produces adequate ozone in consistent quantities over lengthy usages has not been available. Previous small ozone generators, i.e., those creating <one gram of ozone per hour, lose the ability to produce consistent levels of ozone almost immediately when turned on due to heat build up and the subsequent degradation of the ozone output. These small ozone generators are known to lose as much as 80-90% of their rated ozone generation in less than five minutes, leaving the user without the microbial reduction protection necessary for safety. Furthermore, such apparatus have had no means to quantify the presence of sufficient levels of ozone to provide microbial control and no means to alert the user of the ozone apparatus when the apparatus is failing to provide sufficient sanitation levels. Another deficiency in previous corona discharge ozone electrode design has been the need for dry air or for maintenance or cleaning or regular replacement of the electrode due to the build up of nitric acid resulting from moist air feeding the ozone generator. Finally, previous attempts to utilize small ozone generators have lacked the means of delivering the sanitizing oxidant to the point of application in a way that maintains consistency and that provides a means of portability as an optional mode of application.

Examples of prior art ozone generating apparatus include U.S. Pat. No. 5,871,701, and U.S. Pat. No. 5,824,274, both issued in the name of Ronald B, Long, first-named inventor herein, which are incorporated herein by reference in their entirety. The '701 patent teaches the efficacy of small diameter electrode components when utilized for introducing ozone into the air for such purposes as odor removal indoors and similar applications. The '274 patent teaches an apparatus having small diameter electrode components, which are configured so as to be easily removable for cleaning and replacement. U.S. patent application Ser. No. 10/832,401, in the name of Ronald B, Long, first-named inventor herein and which is incorporated herein by reference in its entirety, teaches an apparatus which has a disposable dielectric which required replacement on a regular basis that was accessed from outside the body of the ozone generator by means of a screw cap and an internal manifold which permitted ozone to be directed through different orifices for application in water treatment or in air purification.

### SUMMARY

It is an object of the present invention to provide a greatly enhanced method of ozone generation. The present invention provides consistent levels of ozone regardless of the time span during which the ozone is generated and mixed with water that can then be applied to surfaces of foods and food preparation areas to sanitize them.

The present new and separate invention greatly surpasses the function and design of the small diameter electrodes as described in the aforesaid patents and additionally permits the small diameter ozone generation chamber concept to serve aqueous application of ozone for food and surface sanitizing practices. Also, the present invention eliminates the need for any maintenance whatsoever of the ozone generation chamber with a unique, permanently sealed design which operates at a specifically harmonious rate of electronic frequencies and voltages which prevent the buildup of nitric acid.

The present invention manifests a broad usefulness for sanitizing food and surfaces by combining a compact, economical ozone generation and application apparatus, capable of producing consistent levels of ozone to be mixed with water passing through the apparatus, by utilizing one or more sets of small diameter, sealed ozone generation electrodes, which are individually air-cooled, and which are powered by a combination of frequency and voltage at such harmonic levels as the formation of nitric acid does not occur on the surfaces therein. The apparatus delivers ozone into a stream of water which is directed through a distribution apparatus to the point of application.

In one alternative embodiment, a water quantifier counts the gallons passing through the present invention and provides means through a digital display of the total number of gallons. The display informs the user of the present invention when it is time to change the prefilter to assure that all ozone demand is removed from the process water, thereby assuring the presence of adequate ozone levels in the water to facilitate sanitation of foods and surfaces. It is neither necessary nor desirable that all of the ozone thus delivered be dissolved within the water because the latter is merely the transport agent for the reactive oxidizer. Both microscopic dissolved and somewhat larger entrained bubbles of ozone gas perform the work at the surface of the food and food preparation area. The dissolved ozone, however, provides means for measuring the presence of prescribed levels of ozone and communicating an alert to the apparatus user if the levels fall below minimum design specification of approximately 0.15 parts per million. The dissolved ozone sensor additionally provides means for increasing or, alternatively, decreasing the levels of ozone generated to maintain consistent levels of ozone and for limiting the potential for excessive off-gassing of ozone into the ambient atmosphere of the work area. In one alternative embodiment, cooling air is drawn first through a porous carbon sheet filter to reduce the amount of ambient ozone in the work area adjacent to the present invention. The entire apparatus of the present invention is relatively simple to manufacture and is economical in cost. The apparatus has the added quality of being easily portable and powered by a battery, solar generator or other alternative DC power source.

An apparatus according to one of embodiment of the present invention includes a corona discharge ozone generator, comprising a permanently sealed, concentric anode, dielectric and cathode, for generating ozone, a cooling apparatus connected to the ozone generator for cooling the apparatus during use, tubing connected between an entry port for water and an exit port for water forming a path for the water, an apparatus connected to the ozone generator and the tubing so as to allow a portion of the generated ozone to dissolve in the water passing through the tubing, and a sensor connected along the path for the water, for recognizing levels of oxidant in the water, and adjusting the ozone output by the ozone generator to maintain a predetermined level of dissolved ozone.

The apparatus according to one embodiment includes a water flow quantifier disposed along the tubing for measuring a total volume of water processed, and a display for displaying the measured total volume of water.

The apparatus according to one embodiment includes probes disposed in the water path for sensing levels of dissolved ozone in the water, and alarm devices responsive to and connected to the probes to communicate an unacceptably low level of dissolved ozone in the water.

The corona discharge ozone generator according to one embodiment includes a dielectric glass tube, an anode concentrically encapsulated within the glass tube, a hollow cathode, within which the glass dielectric tube is disposed, annularly in a centered position, a plug connected to a first end of the hollow anode, a fitting having a first end connected to a second end of the hollow anode, the fitting comprising a stepped down orifice extending longitudinally through the fitting, the second end of the hollow anode being configured so as to fit in a large end of the stepped down orifice, and an insulator cap mated to a second end of the fitting.

The apparatus according to one embodiment of the present invention includes a seal placed over a distal end of the anode and forced toward the proximal end to the point in which it communicates with an intersection of the anode and the fitting.

The cooling apparatus according to one embodiment of the present invention includes a cooling fan, a filter mounted at an intake side of the cooling fan, and a cooling fin shaped and connected so as to cover the ozone generator and the cooling fan.

According to one embodiment, the apparatus connected to the ozone generator and the tubing includes a venturi injector having a first passage and a second passage at right angles to the first passage, a first end of the first passage connected to an exit fitting through which the water exits the apparatus, a second end of the first passage connected to a flow switch for controlling flow of the water through the venturi injector, and the second passage connected to the ozone generator.

The apparatus according to one embodiment includes a first alarm indicator which indicates when power is on, a second alarm indicator which indicate when power is initiated to the ozone generator, and a third alarm indicator which indicates if there is a malfunction of the apparatus.

The apparatus according to one embodiment includes an audible alarm generator for generating an audible alarm as long as power is supplied to the ozone generator.

### BRIEF DESCRIPTION OF THE DRAWINGS

For better understanding of the invention and additional objects and advantages thereof, reference is made to the following detailed description and accompanying drawing of a preferred embodiment, wherein:

FIG. 1 shows an exploded view of the apparatus according to one embodiment of the present invention;

FIG. 2 shows the exploded view of the internal configuration of several components related to the ozone generation chamber according to one embodiment of the present invention;

FIG. 3 shows the cross sectional cutaway view of the ozone generation chamber, electrode assembly and the alignment, connection and allocation fitting (ACA);

FIG. 3a shows close-up detail of the ACA fitting shown in FIG. 3;

FIG. 3b shows a close-up view of the arrangement of the anode, dielectric and cathode in communication with the O-ring and cathode plug, shown in FIG. 3;

FIG. 4 shows an overall perspective view of the apparatus according to one embodiment of the present invention;

FIG. 5 shows an exploded view of an alternative embodiment of the present invention; and

FIG. 6 shows an overall perspective view of the alternative embodiment of FIG. 5.

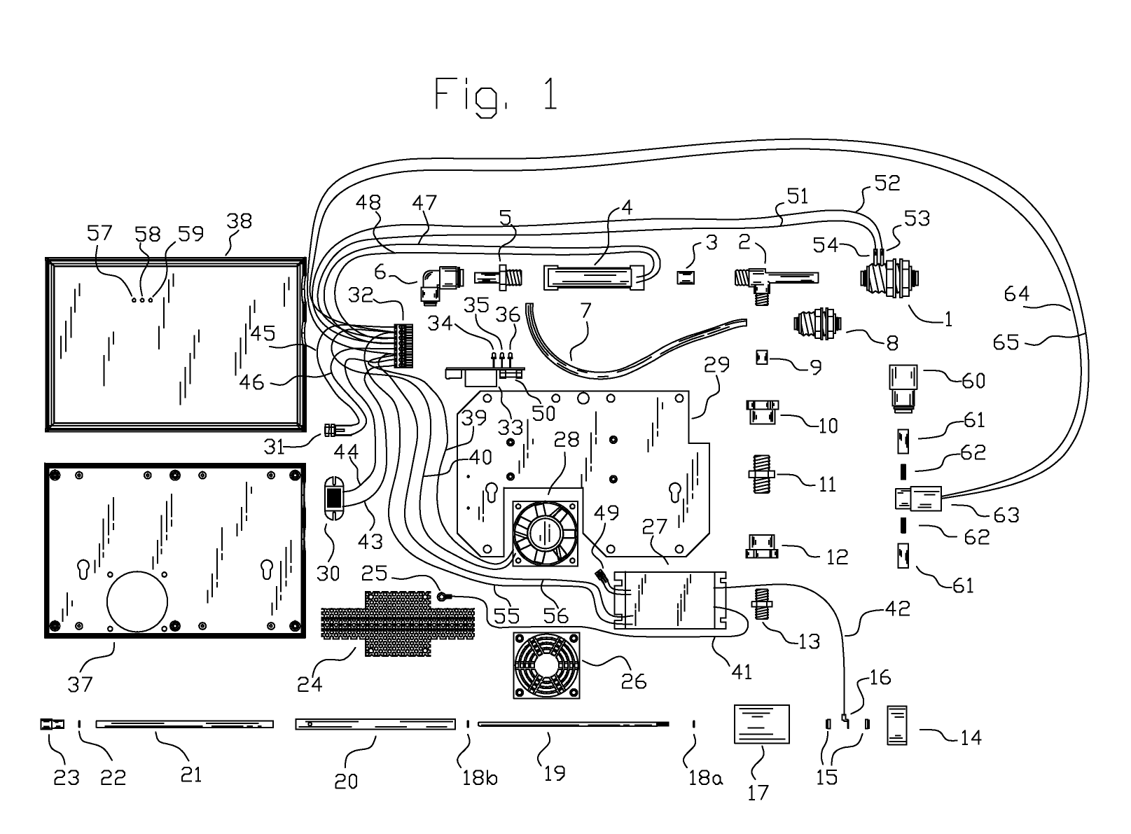

### DETAILED DESCRIPTION

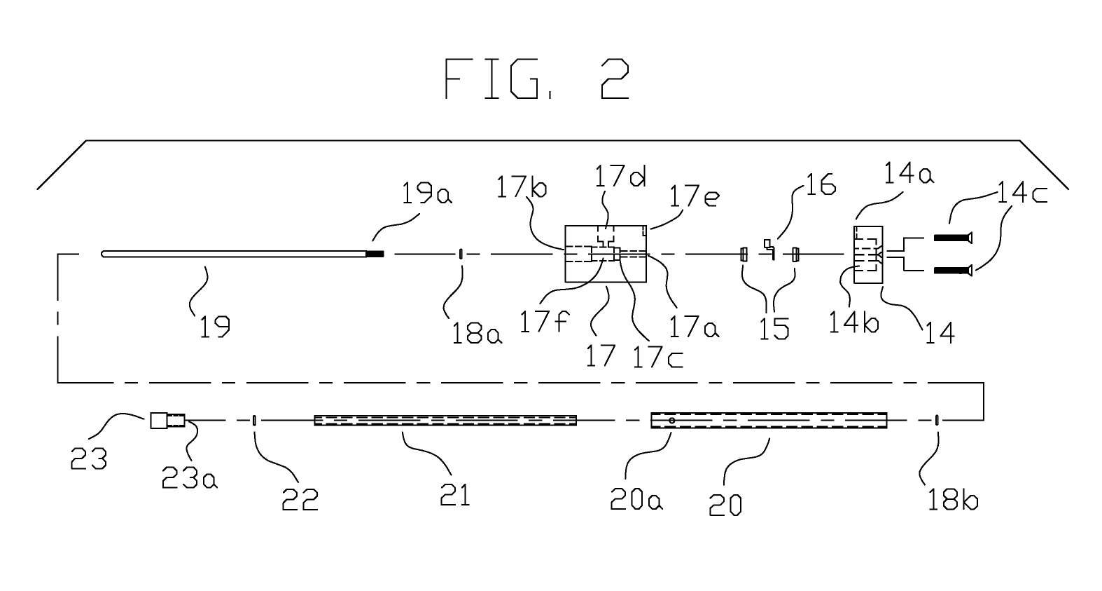

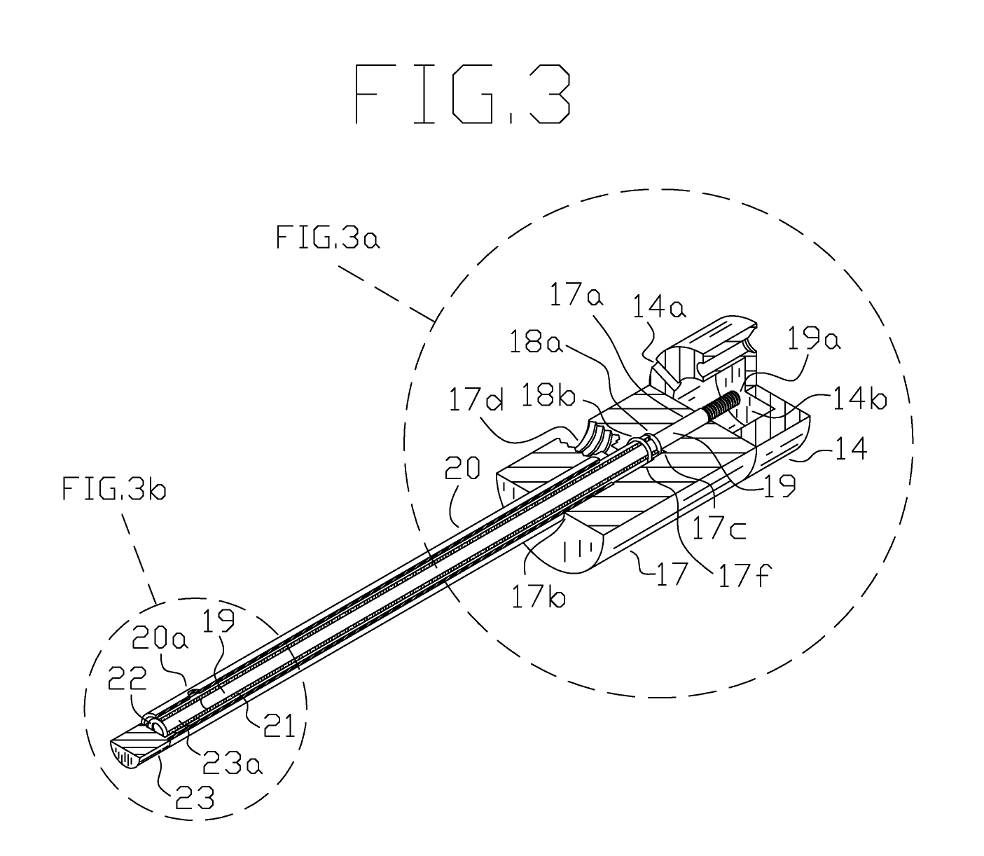

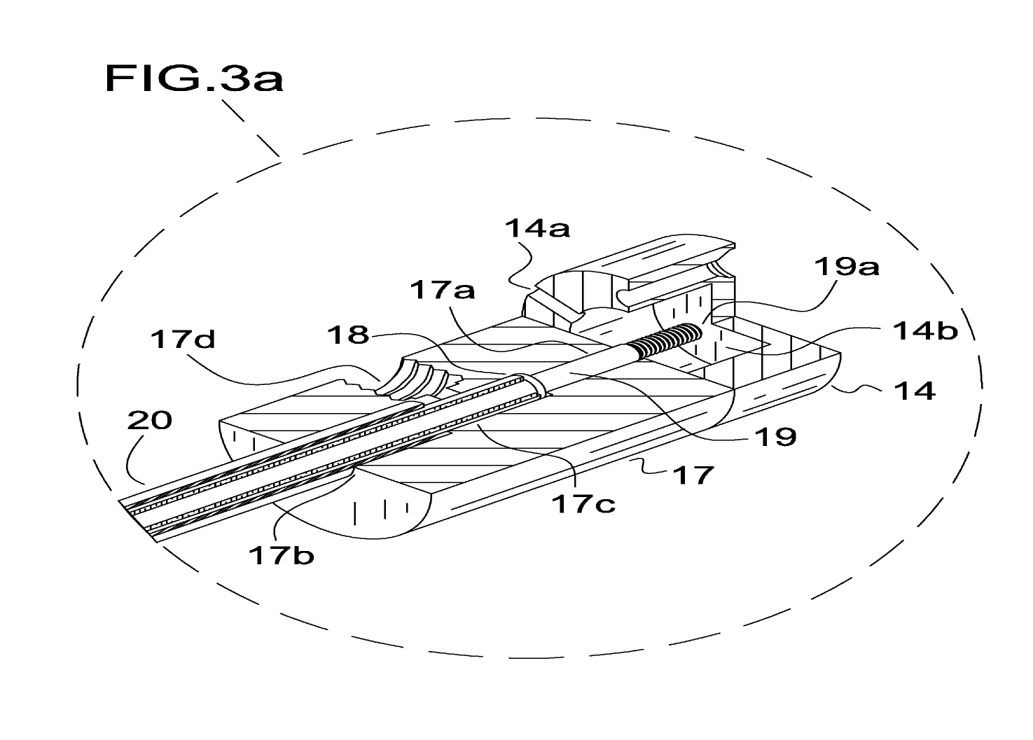

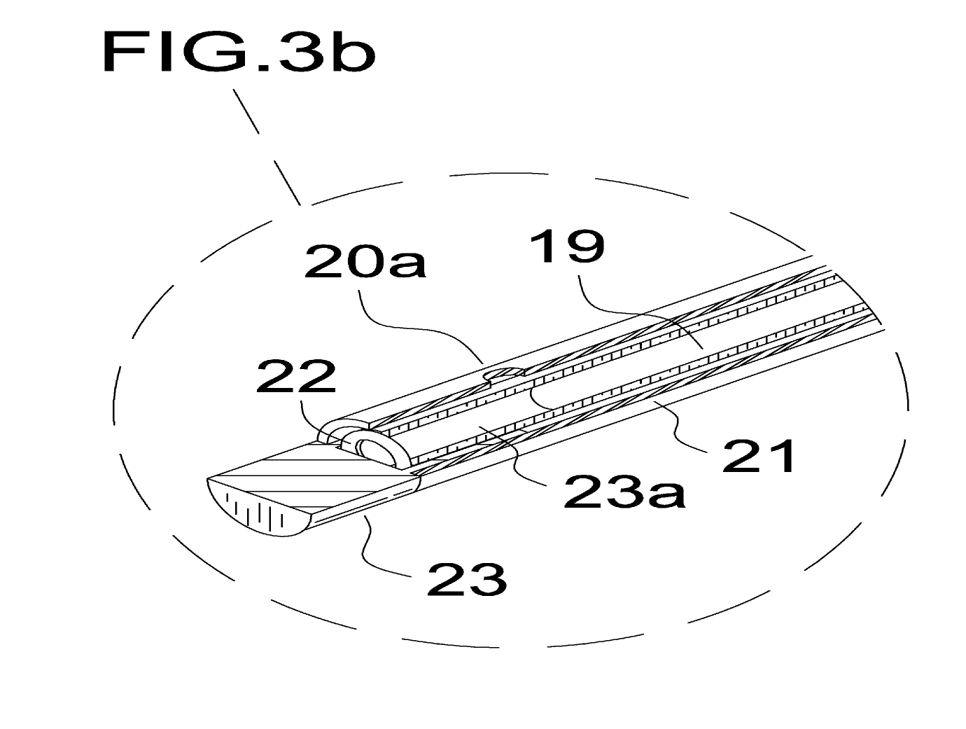

In one embodiment, the small diameter ozone generation chamber (66) is comprised of three components, including an internal anode (19), which is concentrically encapsulated within a tubular dielectric glass (21), which both reside annularly in a centered position inside a hollow cathode (20). The cathode (20) may consist of a tubular length of 316 L polished stainless steel or other suitable conductive and oxidation resistant metal. According to one embodiment, the cathode (20) exhibits a small external diameter of approximately 0.375″, +/−0.005″, and an internal diameter of 0.303″, +/−0.005″. Near the end distal to the ozone exhaust end of the cathode, a small orifice (20a) of approximately 0.125′ is machined into the cathode perpendicular to the length of the cathode (see FIG. 2). As will be described, the location of the machined orifice (20a) is in near proximity to the distal end of the anode so as to permit air flow in to and along the length of the dielectric gap within the chamber (66).

The anode (19) may be formed of a solid length of 316 L stainless steel rod or other similar suitable oxidation resistant metal. According to one embodiment, the anode (19) has a diameter of 0.150″ with tolerance of 0.00″ positive and 0.001″ negative. One end (19a) of the anode is threaded to accept an electrical connection, which according to one embodiment, is comprised of nuts (15) sandwiching a locking ring electrical terminal (16).

The anode (19) closely communicates with an inside surface of the dielectric (21) with a gap tolerance of not more than approximately 0.002″ between the external surface of the anode and the internal surface of the dielectric. The gap entraps ambient air at present atmospheric pressure at the moment of manufacture. The gap between the external surface of the dielectric tube and the polished internal surface of the cathode maintains a tolerance of less than or equal to approximately 0.010″. The entire ozone generation chamber (66) is in communication on its proximal end with an alignment, connection and allocation (ACA) fitting (17), which additionally provides for electrical and gas connection and allocation.

The anode (19) is first machine pressed, threaded end first, through a hole running axially exhibiting a tight frictional fit that secures the anode both longitudinally and latitudinally, thereby preventing its movement in any direction. The threaded end (19a) of the anode is connected to the positive lead (42) of a high voltage, high frequency power supply (27), as described below, by means of a locking ring electrical terminal (16) situated between a first threaded on nut (15a), then the ring connector (16) and finally a locking nut (15b). A single, cushioning o-ring (18) is placed over the distal end of the anode and forced toward the proximal end to the point it communicates with the intersection of the anode (19) and the ACA fitting (17). Said intersection is found inside a female receptacle orifice (17b), which is axially located along the ACA fitting (17), and which provides the secure alignment of the anode (19), as noted above, the dielectric (21) and the cathode (20). According to one embodiment, the female receptacle orifice (17b) is formed of three hollow sections, each section progressively smaller in diameter than the last, i.e., forming a stepped-down orifice (17a-17c) in the ACA fitting (17). The first axial section (17b) has the widest diameter opening, the middle axial section (17c) has the next smallest diameter opening and the third axial section (17a) has the smallest diameter opening. The o-ring (18) provides a cushion for the proximal end of the dielectric that is fitted over the anode (19) and secured by the diameter of the second axial step-down section (17c) in the ACA fitting (17). The o-ring (18a) also provides a seal for the proximal end of the dielectric, affecting the desired sealing of the interior space between the anode (19) and the internal surface cavity of the dielectric (21). A second o-ring (18b) is situated around the body of the dielectric (21) so as to communicate with the step down (170 thereby forming a water tight seal by virtue of its communication with the wall of the ACA fitting (17). The o-ring (18b) is located between the threaded orifice (17d) in the ACA fitting and the step-down (17f) According to one embodiment, the o-rings (18a) and (18b) may be comprised of a highly ozone resistant elastomer material, such as TEFLON® or VITON®, durometer A75, the use of which material is well known for its ozone resistance by practitioners of the art.

The cathode (20) is placed tubularly over the exterior surface of the dielectric (21) and machine pressed into the third step-down section (17b) of the ACA fitting (17) in which the frictional fit between the interior wall of the ACA fitting (17) and the exterior surface of the cathode (20) secures the cathode both longitudinally and lattitudinally. The step-down section (17b) of the ACA fitting (17) is staggered so that the proximal end of the cathode (20) is positioned back away from the proximal end of the dielectric (21), thereby preventing high voltage arcing between the point on the anode (19) at its juncture with the proximal end of the dielectric (21). The ACA fitting section (17b) maintains a solid frictional grasp of the cathode (20) and the dielectric (21) such that there is a uniform dielectric gap along their entire length, providing means for the corona formation and the subsequent formation of ozone gas.

A threaded orifice (17d) in the ACA fitting, perpendicular to the ozone generation electrode or chamber (i.e., the stepped-down orifice (17a-17c)), is located at the point at which the cathode (20) terminates within the ACA fitting (17) in such a manner that ozone gas generated within the length of the dielectric gap may exit the chamber, whether forced by pressure from the distal end of the chamber or drawn out by vacuum exerted at the exterior of the threaded orifice (17d).

A high voltage insulator cap (14) is machined from the same stock as the ACA fitting (17) and mated to its end in diameter and shape opposite the ACA fitting chamber support orifice. The insulator cap (14) has a recessed orifice (14b), which is machined in diameter and depth sufficient to enclose the threaded end wire terminal attachment of the anode (19A, 15, 16). The insulator cap (14) may be formed from an ozone resistant thermoplastic, such as CPVC or similar suitable high dielectric strength material. The insulator cap (14) is fixed to the face of the ACA fitting (17) by means of two fasteners (14c). The positively charged high voltage positive lead (42) from the anode connection is enclosed between two corresponding grooves situated, on one hand, on the lip of the high voltage insulator cap (14a) and, on the other hand, on the face (17e) of the ACA fitting (17). This configuration permits a tight and protective seal around the travel of the high voltage positive lead (42) through the wall comprised of the mated cap (14a) and the ACA fitting face (17e).

The distal end of the ozone generation chamber includes a cathode plug (23), which includes means for centering of the dielectric (21) and sealing of the distal end of the dielectric gap. The plug blank is a dowel of ozone resistant thermoplastic such as CPVC, KYNAR® or TEFLON®, which is approximately the diameter of the outside of the cathode (20). In one embodiment, the exterior diameter of the plug (23) is turned down along a portion of its length so that its one end is approximately 0.001″ larger (nominally 0.304″) than the interior diameter of the cathode for a length of approximately 0.42″. According to one embodiment, the overall length of the plug is approximately 0.85″. According to one embodiment, an interior orifice (23a) of the plug (23) is machined axially to a depth of approximately 0.75″ and an inside diameter of approximately 0.24″, which is approximately the outside diameter of the dielectric (21). A second o-ring (22), identical to the o-ring (18) inserted at the proximal end of the dielectric within the ACA fitting orifice (17c), is inserted into the orifice (23a) of the cathode plug (23) and seated at its extreme end. Like o-ring (18), the second o-ring (22) may be made of a highly ozone resistant elastomer material, such as TEFLON® or VITON®, durometer A75. The cathode plug (23) is pressed into the distal end of the cathode (20) effectively accomplishing, first, the centering of the dielectric (21) within the chamber, second, the sealing of the cathode (20), third, the sealing of the dielectric gap and, fourth, together with the o-ring (18) at the proximal end of the chamber, the cushioning of the dielectric (21), which provides means of protection against breakage during assembly and handling and transit.

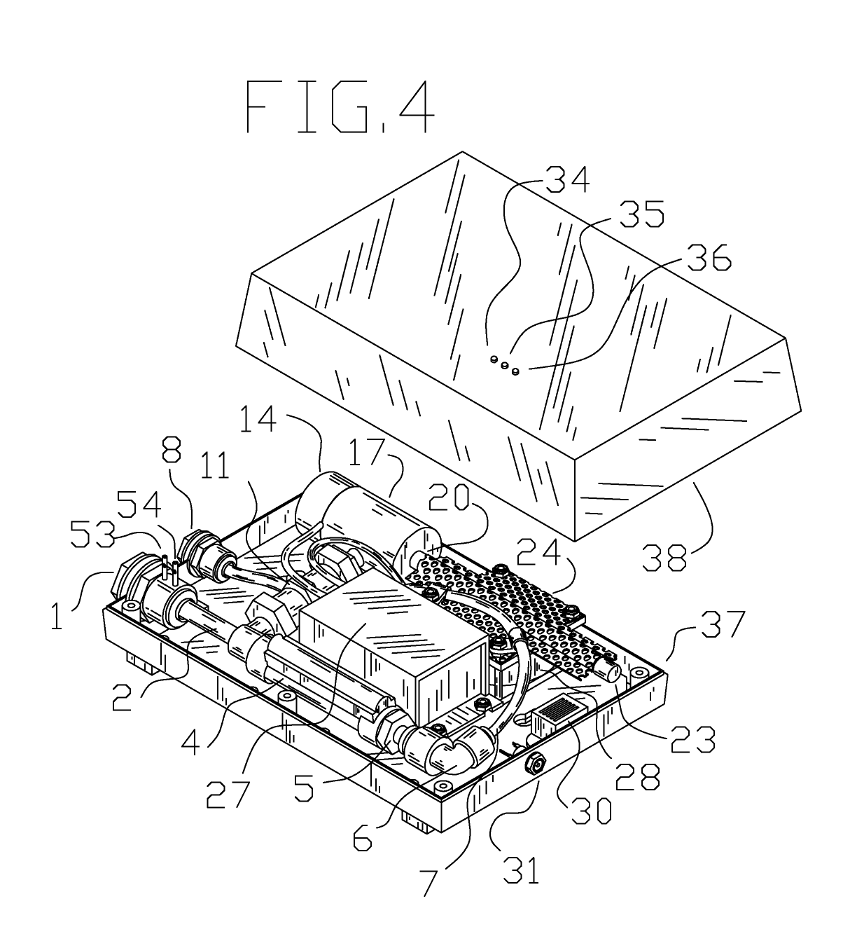

The anode (19), dielectric (21), and cathode (20) arrangement comprising the ozone generation chamber is further in communication along its length with a formed cooling fin (24). According to one embodiment, the cooling fin (24) is composed of a perforated aluminum sheet equal in length to the exposed exterior wall of the cathode and the width of the frame of a small cooling fan (28). A ground wire (41) is connected by means of a ring terminal (25) to the fin (24) from the power supply (27) thereby providing the means of electrical ground for the operation of the corona discharge. The fin (24) must be firmly in communication with the cathode (20) down its length in order to provide grounding to assure the corona forms uniformly. This firm communication is achieved by the force of the four fastener sets (60), each comprised of locking nuts and screw pulling down the fin (24) tightly to the cathode (see FIG. 4). The cooling fan (28) can be any DC voltage fan capable of producing at least 38 cubic feet per minute air flow or more with physical dimensions suitable to fit in the specified space. The cooling fin (24) is formed axially down its longitudinal center line in a semicircular shape or groove that communicates fully down the exposed length of the cathode (20), thereby providing means of transference of heat away from the exterior of the cathode (20). The cooling fin (24) is attached with the four fastener sets (60) to the four corners of the cooling fan, which forces air, drawn from outside the overall apparatus housing through a protective fan screen (26), across the fin (24) and the cathode (20). This construction permits an at least substantially constant reduction in operating temperature and a subsequent consistent level of ozone generation throughout extended time of operation, sufficient by design to maintain full function at predetermined minimum performance levels for the application of ozone to the water. In one alternate embodiment, a granulated carbon sheet filter (61) is mounted at the intake of the cooling fan (28) which assists in the reduction of the ambient ozone in the area of application of the present invention. The carbon sheet filter (61) is held in place by the coverguard (62) of the fan.

Elevated voltages and frequencies are required to generate consistent levels of ozone and the present invention utilizes low input voltages of less than 24 volts direct current from a 120 VAC to, in one embodiment, a 12 VDC converter transformer (not shown). This makes the apparatus inherently safer than typical 120-240 VAC operating apparatus common to many ozone generators. The converter transformer communicates with the components of the apparatus according to the present invention by means of an electrical jack (31), which is mounted into the lower housing (37). Other direct current voltages may be applied in a range from 9VDC to 24VDC. The apparatus may be powered by a DC battery or alternative DC power source, such as solar panel, wind powered electrical generator or similar apparatus. The apparatus may be rendered portable with the addition of a back pack and battery apparatus as well.

The apparatus internal power supply (27) oscillates the direct current back into alternating current while increasing the voltage and frequency substantially to achieve a corona discharge sufficient to produce minimum design levels of ozone. The power supply (27) oscillates the incoming dc power of positive lead (55) and negative lead (56) with an inverting circuit, then it amplifies the oscillating voltage with a voltage amplification circuit causing a high voltage and high frequency output at line (42) and ground wire (41).

For example, in one embodiment, the inverted voltage applied to the corona discharge is in a range, peak to peak, between approximately 5.6 Kv and approximately 8.3 Kv, at a frequency of approximately 94.5K Hertz, typically producing ozone gas in the average concentration range of approximately 4 Grams per cubic meter. (4 G/M₃) at 0° dewpoint.

The electronic characteristics of the present invention include the method for providing an efficient corona discharge and the method for providing operational information through an apparatus of lights and audible alarm (30). In one embodiment, three light emitting diode (LED) lights (34), (35), and (36), are mounted to the body upper cover (38). The first light (34) glows when power is in communication with the apparatus of the present invention from the converter transformer. In one embodiment, this first light would be green in color. A second light (35) flashes when power is initiated to the ozone generation chamber. In one embodiment, this second light would be blue in color. A third light (36) glows red as an alarm indicator if there is a malfunction to the apparatus that results in a failure to operate according to design.

Coupled to the third light (36) by means of a circuit (33) is an audible alarm (30), which is initiated by the same malfunction conditions as the alarm light (35). Both visual (35) and audible (30) alarms will continue until such a time as power is removed from the apparatus by removing the input from the jack (31). In the event of a breakdown of the integrity of the high voltage componentry resulting in an electrical short, in one embodiment, a fuse or breaker (50) with amperage rating of approximately ≦2 Amps shuts down all power to the high voltage components of the apparatus; power to the alarm components, however, remains active.

Additionally provided for is a means of sensing the presence of dissolved ozone in the product water beneath the lower range of design specification, i.e., approximately 0.2 Parts Per Million, and, in the event of failure to maintain said level of minimum acceptable level of dissolved ozone, communicating the lack of performance to the user of the apparatus through the apparatus of audible and visual alarms previously described. The ozone sensing apparatus is a probe set composed of a positive probe (53) and a negative probe (54). The probes (53) and (54) may each be composed of 316 L stainless steel or a suitable alternative material. The probes (53) and (54) are conventional, simple conductive probes. An electrical current is applied and measured between the two probes (53) and (54) inserted in the water stream at (1), resulting from the motion of electrically charged particles in response form the force acting on them from the applied electric circuit (33). A current arises in the water stream from the flow of electrons between probes (53) and (54) causing electronic conduction. When ozone is injected into the water stream at the venture injector (2) the electrical conductivity between probes (53) and (54) become stronger due to the added number of electrons of the ozone molecules that are available to participate to the conduction process. The applying electric circuit (33) then measures this electrical conductivity energy state between the probes and adjusts the power supply (27) with dc current through leads (55) and leads (56), controlling voltage output at leads (41) and (42) maintaining a desired ozone production. The two probes (53) and (54) of the probe set communicate with the stream of water exiting the apparatus of the present invention through a bulkhead fitting (1), which is frictionally in communication with the exit port of a venturi injector (2). The two probes (53, 54) according to one embodiment, are machine pressed through the wall of the bulkhead fitting (1) with their ends extending into the flow of water no more than one half the internal diameter of the proximal throat of the bulkhead fitting (1). In another embodiment, the probes may be in the form of screws which are driven through the wall of the bulkhead fitting. In a third embodiment, the probes may be molded into the bulkhead fitting.

In operation, water is input to the apparatus through the bulkhead fitting (8) and passes through tubing (7) (or tubing (7a), quantifying apparatus (63) and tubing (7b), into elbow (6). From elbow (6), the water passes through a flow switch (4) into venturi injector (2). In venturi injector (2), ozone generated in the ozone generating chamber (66) by the operation of the anode (19), dielectric glass (21) and cathode (20) is dissolved in the water. The ozone is communicated to the venturi injector (2) via the coupler (13), bushing (12), check valve (11), bushing (10), and a stiffening insert (9). Stiffening insert (9) goes into the thin walled intake port of the venturi to support the walls, which are very thin, as bushing 10 is screwed down over insert (9), through the gas intake port (2a). The ozone treated water then exits through the exit bulkhead fitting (1). In an alternative embodiment of the present invention, a normally closed electronic solenoid valve (63) is situated between the ACA fitting (17) and the intake port of the venturi (2) by means of valve fitting adapter (61), which communicates with the body of the solenoid valve (63) by means of threaded valve fittings (62). The distal valve fitting adapter (61) in closest proximity to the venturi (2) communicates with the intake port of the venturi by means of an npt threaded x quick connect adapter (60). In the case of said alternative embodiment of the present invention, the coupler (13), bushing (12) bushing (10) are eliminated. Power to the solenoid valve is transmitted from the power supply via wires (64), (65) when the present invention is activated. Power is supplied to electronic resistance sensing circuitry (not shown) connected at its one side to the positive probe (53) and at its second side to the negative probe (54), which is set to activate both audible and visual alarms, as previously described, if resistivity of the water passing through the exit bulkhead fitting (1) drops by a predetermined percentage set by the manufacturer. If ozone is present in the water stream, electrical resistance of the water, as measured by the probes (53) and (54), increases according to the concentration of the oxidant (e.g., ozone) present therein. Similarly, the electronics of the sensor alters the level of ozone produced to maintain a substantially constant ozone level and to avoid excessive ozone off-gassing from the product water stream.

In one mode of operation, the apparatus generates and applies into the water consistent levels of ozone capable of maintaining in excess of approximately 0.2 Parts Per Million of measurable, dissolved ozone in the water at a variety of flow rates, for example, but not limitation, between approximately 0.5 and 2.0 gallons per minute when most or all ozone demand and competing oxidizers, i.e., chlorine, have been removed from the water by pretreatment such as, but not limited to, sub-micron carbon filtration or reverse osmosis.

The apparatus in this embodiment introduces the ozone into the water by means of a venturi injector (2), which is selected from those injectors commercially available for specific flow rates and which is included in the interior construction or housing of the present invention as further stated. In one alternate embodiment, an automatic shutoff circuit (not shown) has a timer which limits the duty cycle of the apparatus according to the present invention to a predetermined time limit of constant operation. The automatic shutoff circuit may be reset using a reactivation switch.

The ozone gas generated within the ozone generation chamber (66) is communicated to the venturi injector (2) by means a connecting apparatus composed of Polyvinylidene Fluoride, or PVDF, a highly non-reactive and pure thermoplastic fluoropolymer. It is also known as KYNAR®. PVDF is a specialty plastic material in the fluoropolymer family; it is used generally in applications requiring the highest purity, strength, and resistance to solvents, acids, oxidizers, bases and heat with low smoke generation during a fire event. The connecting apparatus is comprised of first a male thread by male thread coupler (13). According to one embodiment, both threaded surfaces are wrapped with TEFLON® tape or coated with TEFLON® thread sealant to assure a leak free fit, as are all threaded fittings in the connecting apparatus. The first end of the coupler (13) is threaded into the previously mentioned female threaded orifice (17d) of the ACA fitting (17). The second end of the coupler (13) is threaded into the smaller orifice of a female by female bushing (12). One end of a male by male check valve (11) is threaded into the larger orifice of the female by female bushing (12) and, the second end of the check valve (11) is threaded into the larger orifice of the female by female bushing (10). The smaller end of the female bushing (10) is threaded onto the male threaded gas intake port (2a) of the venturi injector (2). The commercially available check valve (11) of the connecting apparatus (10-13) includes a seated stainless steel spring (not shown) which supports a stainless steel ball (not shown), thereby maintaining tension pressure permitting the stainless steel ball pressed against a VITON® rubber o-ring seal (not shown), thereby protecting the internal components of the ozone generation chamber from incursion of water both during operation and at rest. The internal workings and configuration of the commercially available check valve are well know to practitioners of the art and thus are not revealed in the drawings. The stainless steel spring of the check valve (10) exhibits a constant nominal pressure of approximately 4.0 inches Hg whenever the ozone generation and application apparatus is static.

The venturi injector (2), which may be one of several commercially available models, is applied according to the desired operating condition, taking into consideration water pressure and flow rate necessary to create a vacuum of approximately 20″ Hg. providing a feedgas rate through the ozone generation chamber of approximately one liter per minute, plus or minus 10%. Water flowing into the venturi injector (2) first passes through, in one embodiment, a flow restrictor (3). The flow restrictor (3) according to one embodiment has an outside diameter approximately equal to +/−0.004″, the inside diameter of the first water entry throat (2b) of the venturi injector (2). The male thread of the venturi water entry throat (2b) communicates with the female water exit throat (4a) of the flow switch (4), which, likewise, may be one of several commercially available models.

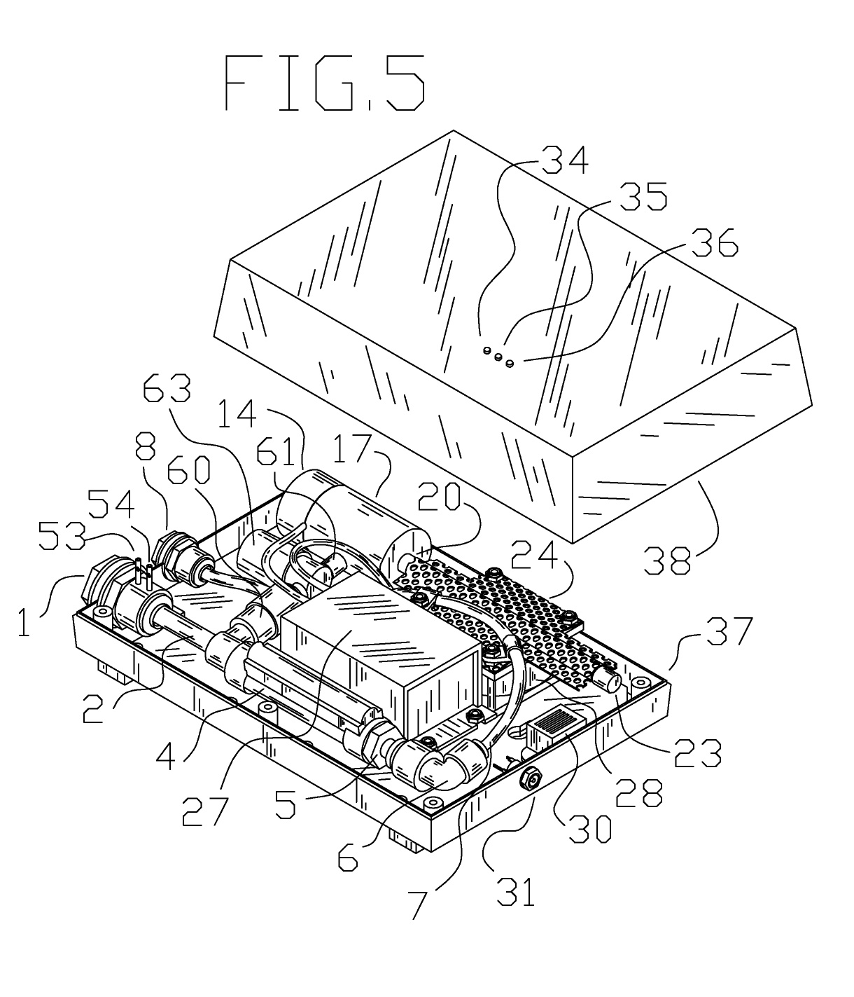

The flow switch (4) provides the method for actuation of the electronic components of the apparatus previously described. Connecting the flow switch (4) to the inflow port of the present invention is, in one embodiment, first, a ⅜″ male thread by ⅜″ male stem (5), in turn, inserted into a ⅜″ female by ¼″ female quick connect elbow reducer (6). A length of flexible tubing (7) is inserted on its first end into the just described elbow reducer (6) ¼″ quick connect (6a) and on its second end into a quick connect ¼″ bulkhead fitting (8) which serves as the entry port for transmission of water through the present invention. According to another embodiment (see FIGS. 5 and 6), a length of flexible tubing (7a) is inserted on its first end into the just described elbow reducer (6) ¼″ quick connect (6a) and on its second end into the entry port of a water flow quantifying apparatus (63), which measures the gallons flowing through it and communicates the data measured to a digital display (64). A second length of flexible tubing (7b) communicates on its first end with the second end of the water flow quantifying apparatus (63) and on its second end to the quick connect ¼″ bulkhead fitting (8). The tubing (7), (7a) and/or (7b) according to the present invention can be made of polyvinylchloride (PVC), or another similar suitable material. In both embodiments, the exit port of water from the present invention is comprised of a ⅜″ quick connect bulkhead fitting (1) into which is inserted, as previously described, in one embodiment, the exit tube portion of the venturi injector (2).

The components, in total, of the present invention are mounted inside, in one embodiment, a two part plastic housing composed of polyethylene, PVC or other similar suitable material. The water contacting components 1 through 8 of the apparatus are held secure in the housing by the frictional clamping of the nuts (not shown) on water entry bulkhead fitting (8) and water exit bulkhead fitting (1). Enumerated components 10 through 26 and the cooling fan (28) are fastened as a unit to the lower housing (37) by means of the four screws and self-locking nuts (not shown) which hold the cooling fan (28), the cooling fin (24) and the ozone generation chamber on the inside and the fan guard (26), which is located exterior to the lower housing (37).

The power supply (27) is affixed to the mounting plate (29) by means of screws and locking nuts (65). Audible alarm (30) is also affixed to the mounting plate (29) with a pair of screws and locking nuts (not shown). The mounting plate (29) is secured to the lower body of the housing (37) by means of screws (not shown) and joined at, in one embodiment six standoffs (not shown), which are molded into the lower body of the housing (37).

Electrical power distribution to the multiple components of the present invention is achieved by means of a plug (32) exhibiting a wiring harness with positive and negative leads to each component. The wiring harness communicates to socket pin connector (32a) and is attached to the circuit board (33). Thus, the positive and negative electrodes (53, 54) of the oxidant sensor (1) communicate with the plug (32) via wires (51, 52); flow switch (4) communicates to socket (32a) via wires (47, 48); input jack (31) communicates to socket (32) via wires (45, 46); audible alarm (30) communicates to socket (32) via wires (43, 44); cooling fan (28) communicates to socket (32) via wires (39, 40); and power supply (27) communicates to socket (32) via wires (55, 56).

Alarm LED lights (34, 35, 36) are connected by solder to the circuit board (33) on their respective power leads, and their domed plastic light covers are affixed to the upper housing component (38) by means of gluing them into three corresponding holes (57, 58, 59).

The foregoing description of the specific embodiments will so fully reveal the general nature of the invention that others can, by applying current knowledge, readily modify and or adapt for various applications such specific embodiments without undue experimentation and without departing from the generic concept, and, therefore such adaptations and modifications should and are intended to be comprehended within the meaning and range of equivalents of the disclosed embodiments. The means and materials for carrying out various disclosed functions may take a variety of alternative forms without departing from the invention. It is to be understood that the phraseology or terminology employed herein is for the purpose of description and not of limitation.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

We claim:

1. An apparatus comprising:
a corona discharge ozone generator, comprising a permanently sealed, concentric anode, dielectric and cathode, for generating ozone;
a cooling apparatus connected to the ozone generator for cooling the apparatus during use;
tubing connected between an entry port for water and an exit port for water forming a path for the water;
an apparatus connected to the ozone generator and the tubing so as to allow a portion of the generated ozone to dissolve in the water passing through the tubing; and
a sensor connected along the path for the water, for recognizing levels of oxidant in the water, and adjusting the ozone output by the ozone generator to maintain a predetermined level of dissolved ozone.

2. The apparatus of claim 1, further comprising:
a water flow quantifier disposed along the tubing for measuring a total volume of water processed; and
a display for displaying the measured total volume of water.

3. The apparatus of claim 1, further comprising:
probes disposed in the water path for sensing levels of dissolved ozone in the water; and
alarm devices responsive to and connected to the probes to communicate an unacceptably low level of dissolved ozone in the water.

4. The apparatus of claim 1, wherein said corona discharge ozone generator comprises:
a dielectric glass tube;
an anode concentrically encapsulated within the glass tube;
a hollow cathode, within which the glass dielectric tube is disposed, annularly in a centered position;
a plug connected to a first end of the hollow anode;
a fitting having a first end connected to a second end of the hollow anode, the fitting comprising a stepped down orifice extending longitudinally through the fitting, the second end of the hollow anode being configured so as to fit in a large end of the stepped down orifice; and
an insulator cap mated to a second end of the fitting.

5. The apparatus of claim 4, further comprising a seal placed over a distal end of the anode and forced toward the proximal end to the point in which it communicates with an intersection of the anode and the fitting.

6. The apparatus of claim 1, wherein the cooling apparatus comprises:
a cooling fan;
a filter mounted at an intake side of the cooling fan; and
a cooling fin shaped and connected so as to cover the ozone generator and the cooling fan.

7. The apparatus of claim 1, wherein the apparatus connected to the ozone generator and the tubing comprises a venturi injector having a first passage and a second passage at right angles to the first passage, a first end of the first passage connected to an exit fitting through which the water exits the apparatus, a second end of the first passage connected to a flow switch for controlling flow of the water through the venturi injector, and the second passage connected to the ozone generator.

8. The apparatus of claim 1, further comprising:
a first alarm indicator which indicates when power is on;
a second alarm indicator which indicate when power is initiated to the ozone generator; and
a third alarm indicator which indicates if there is a malfunction of the apparatus.

9. The apparatus of claim 8, further comprising an audible alarm generator for generating an audible alarm as long as power is supplied to the ozone generator.

10. An apparatus comprising:
a corona discharge ozone generator, comprising a permanently sealed, concentric anode, dielectric and cathode, for generating ozone;
a cooling apparatus connected to the ozone generator for cooling the apparatus during use;
tubing connected between an entry port for water and an exit port for water forming a path for the water;
a venture apparatus connected to the ozone generator and the tubing so as to allow a portion of the generated ozone to dissolve in the water passing through the tubing;
a sensor connected along the path for the water, for recognizing levels of oxidant in the water, and adjusting the ozone output by the ozone generator to maintain a predetermined level of dissolved ozone; and
a housing encapsulating the ozone generator, the cooling apparatus, the tubing, the venturi apparatus, and the sensor.
